(19) United States
(12) Patent Application Publication
(10) Pub. No.: US 2008/0027367 A1
(43) Pub. Date: Jan. 31, 2008

(54) Disposable swab

(76) Inventor: Moshe Abarbanel, Reut (IL)

Correspondence Address: OSTROLENK, FABER, GERB & SOFFEN, LLP, 1180 Avenue of the Americas, New York, NY, 10036, US

(21) Appl. No.: 11/652,927

(22) Filed: Jan. 12, 2007

(30) Foreign Application Priority Data
Jul. 27, 2006 (IL) 177114

(51) Int. Cl. A61M 35/00

(52) U.S. Cl. 604/1

(57) ABSTRACT

An anal swap having a shaft and a tapering head made of concentric layers of cellulose material.

### RELATED APPLICATIONS

Present Application fully incorporates herein by reference and claims all rights of priority to Israeli Patent Application No. 177114, filed on Jul. 27, 2006.

### FIELD OF THE INVENTION

The present invention is directed to a disposable swab, particularly useful for post defecation wiping of the posterior.

### BACKGROUND

Defecation is a necessary if unpleasant fact of life. It is a dirty business that typically leaves residue in the anal region. Removal of such residue is necessary for comfort, hygiene and to avoid medical complaints including rashes, infections and hemorrhoids.

The standard technique for cleaning the anal region is the use of toilet paper which is usually provided in sheet form or on a roll. Toilet paper is advantageous in that it is biodegradable and may be flushed down the sewage pipe with the excrement. However, it has been noted that toilet paper tends to smear excrement over a larger area that the immediate surroundings of the anal orifice. Typically the first wiping spreads the mess and subsequent wiping merely cleans up the mess spread by the first wiping.

Showering or use of a bidet provide a better cleaning technique but are not always practical, requiring permanent plumbing, and cannot be used when camping, for example.

Over the ages, different techniques for this intimate cleaning have been used in different cultures. These include mosses, hay, sticks and stones.

In the developed world, toilet paper is almost universally adopted for cleaning the posterior, indicating inherent disadvantages of earlier techniques that have lost favour. Such early techniques including the gompf; a stick used in medieval privies that was apparently shared between the patrons.

There present invention addresses the removal of excrement from the anal region, and proposes a new device for so doing.

### SUMMARY OF THE INVENTION

It is an aim of preferred embodiments of the invention to provide a disposable device for post defecation cleaning of the anal region.

It is a further aim of preferred embodiments of the present invention that the device is economical to manufacture.

It is a further aim of preferred embodiments of the present invention, that the device be ecologically friendly.

In a first aspect, the present invention is directed to providing an anal swab comprising a shaft and a tapering head of concentric layers of cellulose laminate material.

Preferably the anal swab comprises a rolled up strip of a material selected from the list of papers and cardboards.

Typically the tapering head is integral with the shaft and is fabricated by telescopically rolling the strip of a material.

Alternatively, the anal swab is extruded through a die or pressure molded in a closed mold.

Preferably the anal swab further comprises a biodegradable binder.

Most preferably the biodegradable binder is readily soluble in water.

Such a biodegradable binder is optionally selectable from the list of gelatin, starch and agar.

Optionally at least part of the tapering head is impregnated with an emollient. Optionally the emollient comprises active ingredients for treatment of medical complaints selected from the list of rashes, hemorrhoids and infections. In some embodiments, the emollient comprises active ingredients selected from the list of fungicides and bactericides. Optionally the emollient comprises zinc oxide. Additionally or alternatively emollient comprises petroleum jelly.

In one embodiment the tapering head of the anal swab is fabricated separately from the shaft.

Optionally the tapering head is angled to the shaft.

In one embodiment, the tapering head is coupled to the shaft by a neck having a higher degree of flexibility than the head or the shaft.

In a second aspect, the present invention is directed to providing a dispenser for the anal swabs comprising a shaft and a tapering head of concentric layers of cellulose laminate material.

Optionally the dispenser comprises a box with fixing means for fixing to a toilet roll holder.

In a third aspect, the present invention is directed to providing a method for cleaning the anus comprising removal of excretal remains by scooping up and collecting the excretal remains within the layers of the head of an anal swab and providing a post scooping wipe by pressing the anal swab against the anal region; the anal swab comprising a shaft and a tapering head of concentric layers of cellulose laminate material.

Preferably the method further comprises a post scooping wipe with a laminar wipe selected from the list of toilet paper and wet wipes.

### BRIEF DESCRIPTION OF THE FIGURES

For a better understanding of the invention and to show how it may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention; the description taken with the drawings making apparent to those skilled in the art how the several forms of the invention may be embodied in practice. In the accompanying drawings:

FIG. 1 is a schematic isometric projection of an anal swab in accordance with a first embodiment of the present invention;

FIG. 2 is a schematic isometric projection of an anal swab in accordance with a second embodiment of the present invention;

FIG. 3 is a schematic isometric projection of an anal swab in accordance with a third embodiment of the present invention;

FIG. 4 is a schematic isometric projection of the anal swab of FIG. 3 from a different viewing angle;

FIG. 5 is a side view of a posterior showing the anal swab of the invention in use;

FIG. 6 is a view of the buttocks and anus from below, showing how the tool of the anal swab of the invention provides directed, focussed cleaning of the anal region;

FIG. 7 shows a fourth embodiment wherewith the tapering head of the swab may be angled to the shaft thereof by means of a flexible handle;

FIG. 8A to 8F show different configurations for anal swab heads, and

FIG. 9 shows a dispenser for anal swabs that may be connected to a toilet roll holder.

### DESCRIPTION OF THE PREFERRED EMBODIMENTS

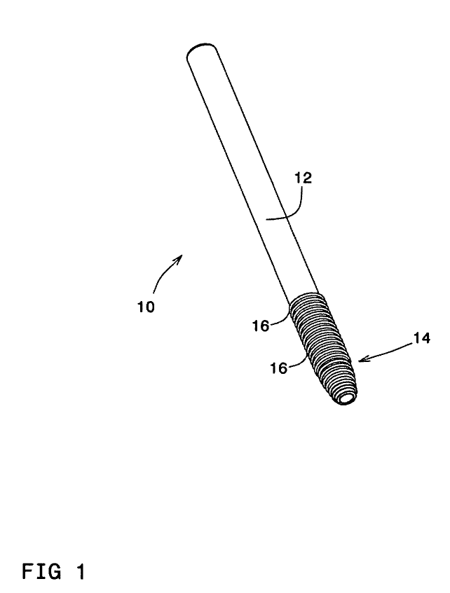

With reference now to FIG. 1, a first embodiment of the present invention consisting of an anal swab 10 is shown in isometric projection. Anal swab 10 comprises a shaft 12 and a tapering head 14 consisting of concentric layers 16.

Typically the tapering head 14, and often the shaft 12 of anal swab 10 are fabricated from a cellulose based laminate material, such as paper or cardboard, for example. Consequentially the swab 10 is easily disposable, and preferably fully flushable down the toilet and biodegradable.

The anal swab may have the approximate dimensions of a cigarette, tough typically the shaft will be a little longer. There is a great deal of flexibility in the dimensions thereof, but it is typically 7 to 15 cm long and 7 to 15 mm in diameter.

The anal swab is typically fabricated from a one or multi-ply strip of the cellulose laminated material, such as tissue paper, for example, and may include a biodegradable binder such as gelatin, starch or agar.

The anal swab 10, or at least part of the tapering head 14 thereof, may be impregnated with an emollient. An appropriate emollient may include a range of active ingredients for treatment or prevention of minor medical complaints such as rashes, hemorrhoids and infections, for example. In some embodiments, the emollient comprises active ingredients selected from the list of fungicides and bactericides. Optionally such an emollient may comprise zinc oxide as widely used in baby creams. The emollient may also/alternatively include petroleum jelly.

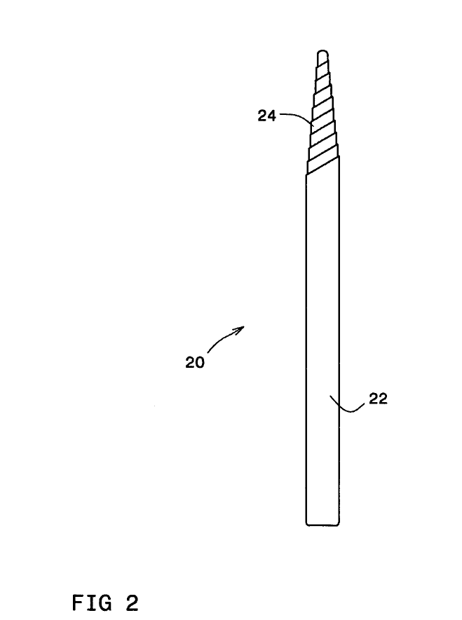

FIG. 2 is a schematic isometric projection of an anal swab 20 in accordance with a second embodiment of the present invention where the shaft 22 and tapering head 24 are fabricated of concentric layers of cellulose laminate material. Typically, such anal swabs 20 are produced by rolling up single or multiple ply cellulose laminar materials such as paper or card. Miss-rolling a rectangular strip or starting from the long parallel edge of a trapezium shaped strip of cellulose laminar material conveniently provides a tapering head 24 that is integral with the shaft 22.

Other fabrication techniques are possible however. These include, inter alia, extrusion through a die, and molding in a closed mold.

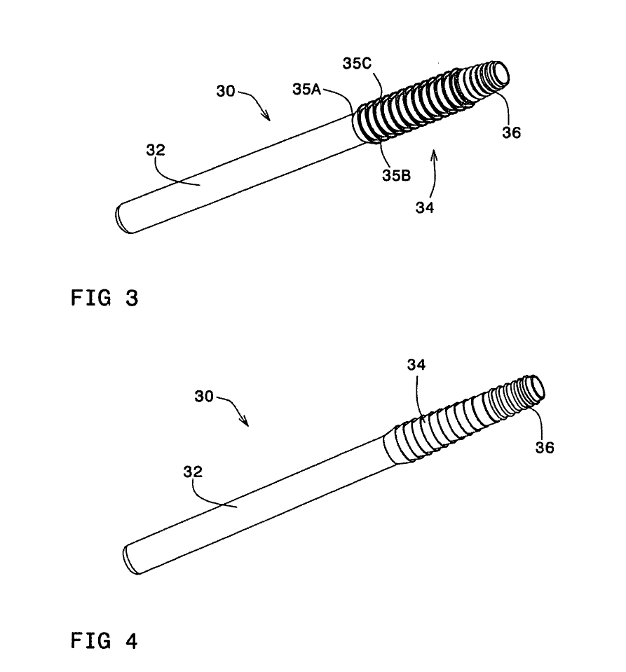

FIG. 3 and FIG. 4 show an anal swab 30 in accordance with a third embodiment of the present invention wherein the proximal most concentric layers 35A, 35B, 35C of the head 34 are widened out and distal edges thereof protrude outwards from the diameter of the shaft 32. The front section 36 of the swab 30 head 34 tapers inwards to a blunt tip. Such an anal swab 30 may be fabricated by rolling up a strip of cellulose laminar material and then tamping the front section, perhaps into a socket. Alternatively, the swab may consist of a telescopic stack of independent layers that are co extruded through a die.

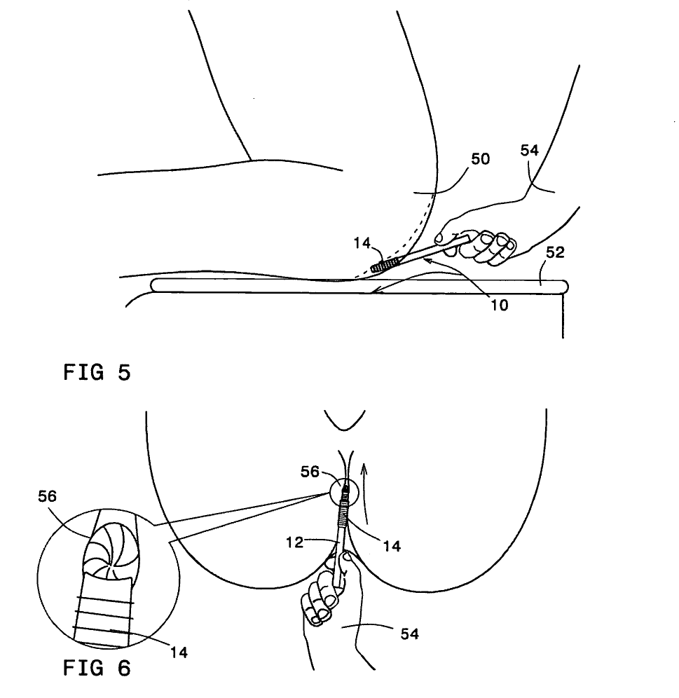

FIG. 5 and FIG. 6 show, in side view and horizontal section from below, the posterior (buttocks) 50 of a person sitting on a toilet seat 52 showing how an anal swab 10 of the invention may be held by the shaft 12 thereof in the hand 54 of the person and used to provided directed, focussed cleaning of the anus 56 and region therearound.

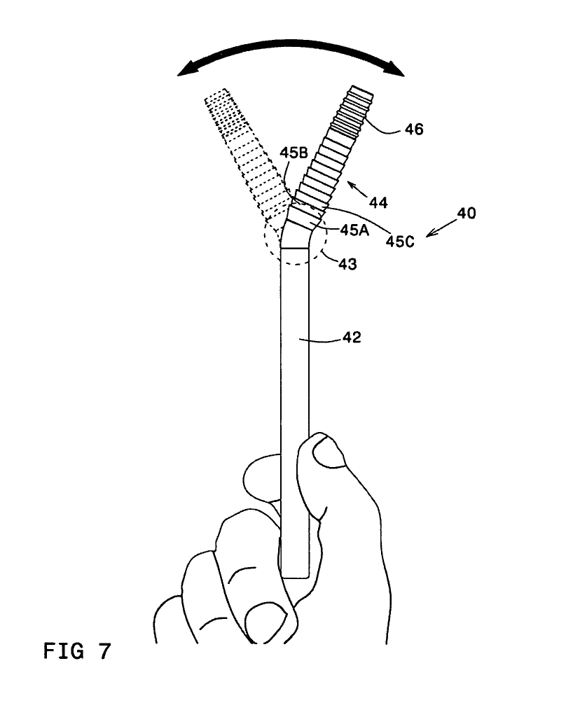

The anal swab is capable of some variation, for example, FIG. 7 shows a fourth embodiment of the anal swab 40 wherewith the tapering head 44 of the swab 40 may be angled to the shaft 42 thereof by means of a bent neck 43. The design of the head 44 may include concentric, flared stepped layers 45A, 45B, 45C and a tapering, typically stepped tip 46. Such an anal swab 40 may be particularly useful for a user with limited mobility, for example.

Preferably the anal swab provides a soft scrubbing tactile sensation, but is sufficiently stiff to scoop up solid waste.

In one embodiment, the tapering head 44 is coupled to the shaft 42 by a neck 43 having a higher degree of flexibility than the head 44 or the shaft 42.

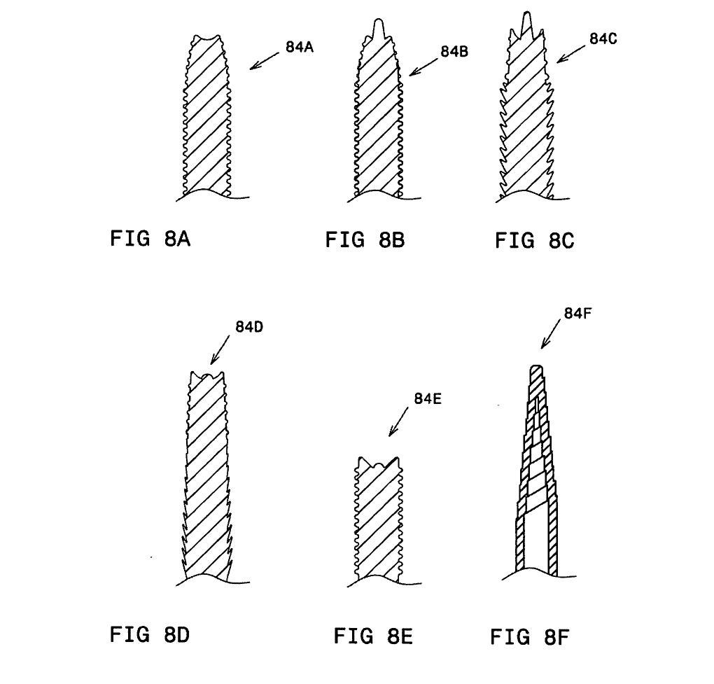

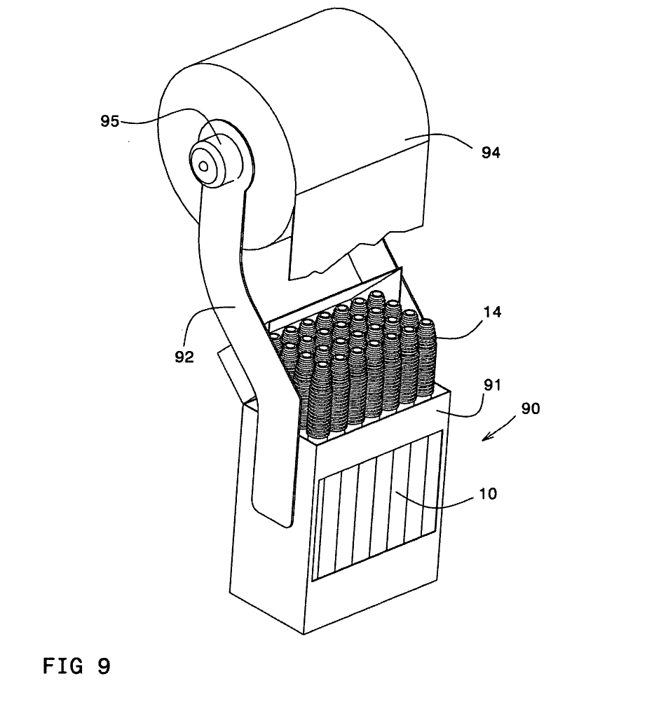

FIG. 8A-8F, show a variety of tapering heads 84A-84F that have been tested. It is not yet known which head type is optimal, and this may be a matter of personal choice, though clearly influenced by the consistency of the stool, shape of anus, hemorrhoids, and the like. The various tapering heads 84 may be cofabricated with the shaft of the anal swab or retrofitted to a separately fabricated shaft.

FIG. 9 shows a dispenser 90 for anal swabs 10 that consists of a box 91 connected to a toilet roll holder 95 by a connection 92. Conveniently, the dispenser 90 hangs beneath a roll of toilet paper 94 for dispensing sheets of toilet paper for final wiping, for mopping up liquid excretions, and the like.

Thus a new personal hygiene device is disclosed, that is particularly useful for cleaning the anal region after defecating. The method for cleaning the anus therewith includes the step of gouging up and collecting excretal remains within the layers of the head 14 of an anal swab 10 by scooping up the excretal remains by pressing the anal swab against the anal region 56, typically providing a post scooping wipe by pressing the anal swab against the anal region which may be followed by a subsequent wiping effect, using conventional toilet paper, for example. The anal swab 10 comprises a shaft 12 and a tapering head 14 of concentric layers of cellulose laminate material.

Preferably, for final cleaning, the method further comprises a post scooping wipe with a laminar wipe such as toilet paper 94 or wet wipes. For best results, a user may use a wet wiping product (e.g., wet toilet paper or wet wipes) for the post scooping wipe and follow up with a dry laminar wipe, for example dry toilet paper.

Thus the scope of the present invention is defined by the appended claims and includes both combinations and sub combinations of the various features described hereinabove as well as variations and modifications thereof, which would occur to persons skilled in the art upon reading the foregoing description.

In the claims, the word “comprise”, and variations thereof such as “comprises”, “comprising” and the like indicate that the components listed are included, but not generally to the exclusion of other components.

What is claimed is:

1. An anal swap comprising:
a shaft; and
a tapering head having concentric layers of cellulose material.

2. The anal swab of claim 1 wherein said shaft and said tapering head are integrally formed from a rolled up strip of said cellulose material.

3. The anal swab of claim 2 wherein said tapering head is formed by telescopically rolling the strip of said material.

4. The anal swab of claim 1, wherein said swab is fabricated by extrusion through a die.

5. The anal swab of claim 1, wherein said swab is fabricated by molding in a closed mold.

6. The anal swab of claim 1, wherein said swab is capable of being flushed down a toilet.

7. The anal swab of claim 1 further comprising a biodegradable binder.

8. The anal swab of claim 7, wherein the biodegradable binder is readily water soluble.

9. The anal swab of claim 7 wherein the biodegradable binder is selected from the list of gelatin, starch and agar.

10. The anal swab of claim 1 wherein at least a part of the tapering head is impregnated with an emollient.

11. The anal swab of claim 10 wherein said emollient comprises active ingredients for treatment of medical complaints selected from the list of rash, hemorrhoids and infection.

12. The anal swab of claim 10 wherein said emollient comprises active ingredients selected from the list of fungicide and bactericide.

13. The anal swab of claim 10 wherein said emollient comprises zinc oxide.

14. The anal swab of claim 10 wherein said emollient comprises petroleum jelly.

15. The anal swab of claim 1 wherein said tapering head is fabricated separately from said shaft.

16. The anal swab of claim 1 wherein said tapering head is angled with respect to said shaft.

17. The anal swab of claim 1 wherein said tapering head is coupled to said shaft by a neck having a higher degree of flexibility than said tapering head or said shaft.

18. A method for cleaning the anus, comprising the steps of
removing excretal remains by scooping up and collecting the excretal remains within layers of a tapering head of an anal swab; and
providing a post scooping wipe by pressing the anal swab against the anal region; the anal swab comprising a shaft and said tapering head having concentric layers of cellulose material.

19. The method of claim 18 wherein said post scooping wipe is accomplished using a laminar wipe selected from the list of toilet paper and wet wipe.
